(19) United States
(12) Patent Application Publication
(10) Pub. No.: US 2022/0226154 A1
(43) Pub. Date: Jul. 21, 2022

(54) SWIM GOGGLE

(71) Applicant: FINIS INC, Livermore, CA (US)

(72) Inventor: David F. Beittel, San Leandro, CA (US)

(73) Assignee: FINIS INC, Livermore, CA (US)

(21) Appl. No.: 17/803,036

(22) Filed: Jan. 19, 2022

Related U.S. Application Data
(60) Provisional application No. 63/205,957, filed on Jan. 20, 2021.

(51) Int. Cl. A61F 9/02; A63B 33/00

(52) U.S. Cl. CPC A61F 9/027; A63B 33/002

(57) ABSTRACT

A goggle with a soft head strap having a circular cupping or open bucket portion is attached a goggle frame with one or more lenses through an attachment mechanism. The attachment mechanism includes brackets attached to strap ends and having matched apertures that press or snap onto raised clip structures on side portions of the goggle frame.

### RELATED APPLICATION

This application claims priority under 35 U.S.C. § 119(e) from the co-pending U.S. Provisional Patent Application Ser. No. 63/205,957, filed on Jan. 20, 2021 and titled “GOGGLE.” The co-pending U.S. Provisional Patent Application Ser. No. 63/205,957, filed on Jan. 20, 2021 and titled “GOGGLE” is hereby incorporated by reference.

### FIELD OF THE INVENTION

The invention relates to swimming equipment. More particularly, the present invention relates swim goggles.

### BACKGROUND

Swim goggles are used to protect a swimmers eyes while swimming or diving. A soft strap that encircles a portion of the swimmers head is useful for keeping the goggles securely positioned and over the swimmers eyes.

### SUMMARY OF INVENTION

The present invention is directed to an attachment mechanisms for securing a goggle frame with one or more lenses to a soft head strap that encircles a portion of a swimmers head. A goggle with a soft head strap that encircles a portion of a swimmers head is described in U.S. Pat. No. 8,448,267 to Hahn.

In accordance with the embodiments of the invention, the attachment mechanism includes brackets with slotted features for attaching to strap ends. The strap ends can include two-part hook and loop fabric, wherein portions of the end straps thread through the slotted features of the brackets and then fold back onto themselves with adjacent surfaces of the strap ends having pieces of the two-part hook and loop fabric attached thereto.

In an alternative embodiments of the invention, portions of strap ends are secured to or through the slotted features of the brackets through two-part snap features, elastic bands, clips or any other suitable means.

In accordance the embodiments of the invention a goggle frame that includes one or more lenses has raised clip structures side portions of the goggle frame. The raised clip structures can have any suitable shape but are preferably substantially round in circumference. The brackets include apertures that also have any suitable shape including, but not limited to, round, oval, triangular, rectangular and star-shaped, but are preferably “matched” to the circumference shape of the raised clip structures.

In operation the brackets with the strap ends attached thereto, such as described above, are debatably attached to the goggle frame by placing the matched apertures on the brackets over the raised clip structures and are then pressed onto and/or rotated onto the to raised clip structures to thereby secure the clip structures, and thus the strap ends, onto the goggle frame.

While the strap ends can be attached any type of head strap, preferably the strap ends are part of a soft head strap with a circular cupping or open bucket portion that encircles a back portion of a swimmer's head with the goggle frame and goggle lens or lenses secured over the swimmer's eyes.

### BRIEF DESCRIPTION OF THE DRAWINGS

FIG. 1 shows a schematic representation of an attachment mechanism for attaching a head strap to a goggle frame, in accordance with the embodiments of the invention.

FIGS. 2A-C illustrates views of a goggle with a head strap attached to goggle frames, in accordance with the embodiments of the invention.

### DETAILED DESCRIPTION OF THE INVENTION

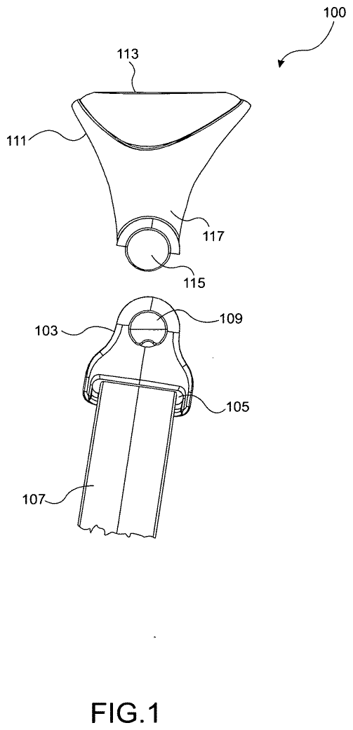

Referring to FIG. 1, a goggle includes an attachment mechanism 100 for attaching a head strap end 107 to a goggle frame 111 through a bracket 103. The bracket 103 has a slotted feature 105 for attaching to the head strap end 107 to the bracket 103. The head strap end 107 can include two-part hook and loop fabric, wherein a portion of the head strap end 107 is thread through the slotted feature 105 of the bracket and is folded back onto itself with adjacent contact surfaces of the strap end 107 having pieces of the two-part hook and loop fabric attached thereto. In addition to, or in place of, two-part hook and loop fabric, the head strap end 107 can be secured to the bracket 103, or through the slotted feature 105 of the bracket 103, through two-part snap features, elastic bands, clips or any other suitable means. The bracket 103 also has an aperture aperture 109 that is shown here as being substantially round, but can have any suitable shape.

The attachment mechanism 100 also includes a raised clip feature 115 that is attached to a side portion 117 of a goggle frame 111. The goggle frame 111 also has one or more lenses 113 attached thereto. The raised clip structures 115 can have any suitable shape but is matched to the shape of the aperture 109 on the bracket 103 such that the aperture 109 can be placed over the raised clip structure 115 and pressed on to raised clip structure 115 and/or rotated on to raised clip structure 115 to secure the clip structure and the bracket 103 together, and thus secure the head strap end 107 on to the goggle frame 111.

In an alternative embodiment of the invention a raised clip structure, such as the raised clip structure 115, protrudes from a surface of the bracket 103 and is configured to engage a matched aperture, such as the aperture 109, positioned on a side surface 117 of the goggle frame 111 to achieve attachment of the head strap end 107 to the goggle frame 111.

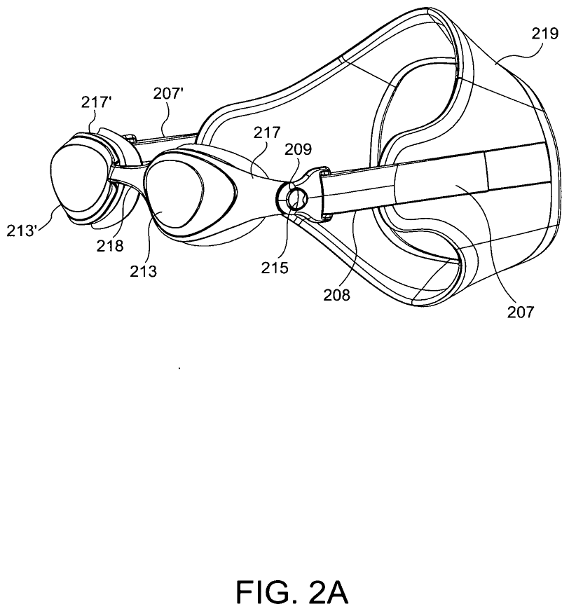

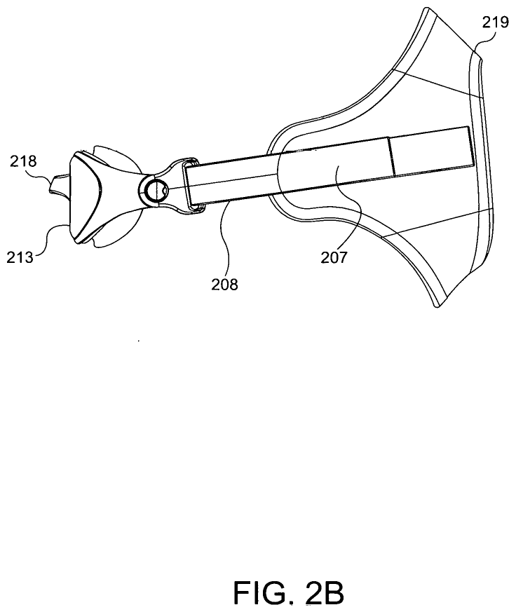

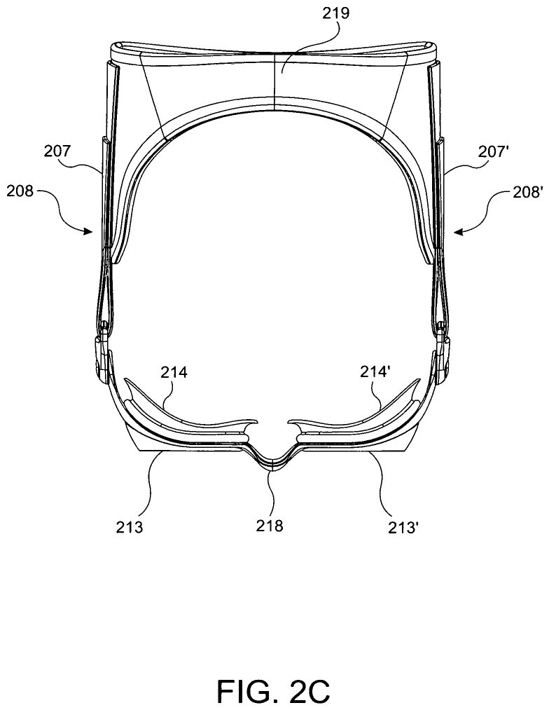

Referring now to FIGS. 2A-C a goggle includes lens frames 211 and 211′ for holding lenses 213 and 213′. The lens frames 211 an 211′ are secured together through a nose bridge 218. Preferably, the lens frames 211 and 211′ also include flexible skirts 214 and 214′ for creating water-tight seal with the frames 211 and 211′ positioned over eyes of a swimmer and secured to the head of the swimmer with a head strap, such as described below.

The goggle includes attachment mechanism 201 and 201′ on each side of the goggle for attaching head strap ends 207 and 207′ to the goggle frames 211 and 211′ through brackets 203 and 203′. The brackets 203 and 203′ each have a slotted features 205 and 205′ for attaching to the head strap ends 207 an 207′ to the brackets 203 and 203′. The head strap ends 207 and 207′ can include two-part hook and loop fabric, wherein portions 208 and 208′ of the head strap ends 207 and 207 thread through the slotted features 205 and 205′ of the brackets 203 and 203′ and are folded back onto itself with adjacent contact surfaces of the strap ends 207 and 207′ having the pieces of the two-part hook and loop fabric attached thereto. In addition to, or in place of the two-part hook and loop fabric, the head strap ends 207 and 207′ can be secured to the brackets 203 and 203′ or through the slotted feature 205 and 205′ of the bracket 203 and 203′ using two-part snap features, elastic bands, clips or any other suitable means. The brackets 203 and 203′ also have apertures 209 and 209′ that engage with and attach to raised clip feature 215 and 215′ that are attached to and protrude from side portions 217 and 217′ of the goggle frames 211 and 211′. The raised clip structures 215 and 215′ can have any suitable shapes but are matched to the shape of the aperture 209 and 209′ such that the apertures 209 and 209′ can be placed over the raised clip structures 215 and 215′ and pressed on to the raised clip structures 215 and 215′ and/or rotated on to the raised clip structure 215 and 215′ to thereby secure the brackets 203 and 203′, and thus the strap ends 207 and 207′ to the goggle frames 211 and 211′.

Attached to the strap ends 207 and 207′ is a soft head strap with a circular cupping or open bucket portion 219 that encircles a back portion of a swimmers head with the goggle frames 211 and 211′ and the lenses 213 and 213 secured over and in front of a swimmers eyes.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. A goggle comprising:
a) brackets attached to head strap ends, the brackets having apertures; and
b) raised clip structures on side portions of a goggle frame, wherein the shapes of the of the apertures and the raised clip structures are matched, such the brackets are debatably attached to the goggle frame but placing the apertures over the raised clip structures and pressing and/or rotating brackets onto the clip structures securing the strap ends to the goggle frame.

2. The goggle of claim 1, wherein the apertures and the circumferences of the raised clip structures are substantially round.

3. The goggle of claim 1, wherein the head strap ends are attached to a soft head strap with a circular cupping or open bucket portion.

4. The goggle of claim 1, wherein the head strap ends are attached to the brackets through slotted features that the head strap ends are treaded through and folded back onto themselves.

5. A goggle with an attachment mechanism for attaching head strap ends to a goggle frame, the attachment mechanism comprising brackets that couple to the strap ends and that have apertures and raised clip structures protruding from sides of the goggle frame with matched circumference shapes to that of the apertures, wherein pressing and or rotating the brackets over the raised snap features secures the brackets to the goggle frame.

6. The goggle of claim 5, wherein the head strap ends are attached to a soft head strap with a circular cupping or open bucket portion.

7. The goggle of claim 5, wherein the head strap ends are attached to the brackets through slotted features that the head strap ends thread through and folded back onto themselves.

8. A goggle comprising:
a) brackets with slotted features for attaching to ends of a strap to the brackets, the brackets further having substantially round apertures; and
b) a frame with one or more lenses and having substantially round raised clip structures on side portions of the frame, wherein the substantially round raised clip structures detachably snap into the substantially round apertures of the brackets to thereby assemble the goggle.

9. The goggle of claim 8, wherein the strap ends are attached to a soft head strap with a circular cupping or open bucket portion.
